(19) United States
(12) Patent Application Publication
(10) Pub. No.: US 2018/0250445 A1
(43) Pub. Date: Sep. 6, 2018

(54) IMPLANTS

(71) Applicant: CeramTec GmbH, Plochingen (DE)

(72) Inventors: Heinrich WECKER, Eckental (DE); Alfons KELNBERGER, Röthenbach (DE); Frank ZIERMANN, Berlin (DE)

(21) Appl. No.: 15/755,949

(22) PCT Filed: Aug. 31, 2016
(86) PCT No.: PCT/EP2016/070482

§ 371 Date: Feb. 27, 2018

(30) Foreign Application Priority Data
Aug. 31, 2015 (DE) 10 2015 216 641.4

(51) Int. Cl. A61L 27/56; A61L 27/32; A61L 27/30

(52) U.S. Cl. CPC A61L 27/56; A61L 27/32; A61L 2430/24; A61L 2420/08; A61L 2430/02; A61L 27/306

(57) ABSTRACT

The present invention relates to implants, methods for the production thereof and the use thereof.

The present invention relates to implants, to methods for the production thereof, and to the use thereof.

Implants for the partial restoration of joint surfaces are known and are implanted when irreparable damage to cartilage, for example due to osteoarthritis, is present in a wide variety of joints in the human body and leads to impairment. Once joint surfaces have become damaged or destroyed by wear, disease or injury, for example, and neither conservative non-surgical treatment methods nor joint-preserving procedures promise successful healing, the implantation of a partial prosthesis generally becomes necessary. These partial endoprosthetic implants are considered to be a treatment concept for patients for whom a full endoprosthesis would be too invasive or is not an option due to the patient's active lifestyle. The goal is to thereby eliminate or reduce the pain in the affected joint, so as to increase mobility and the quality of life in the daily routine, profession and spare time, as well as restore maximum mobility of this joint to as great an extent as possible.

In contrast to total endoprostheses, only a portion of the articular surface is restored with this approach. The required procedures can generally be minimally invasive.

At the same time, the potential of surgery for a total endoprosthesis is preserved. This may be done at a later point in time. This may also benefit older patients, for example if they reject a total joint replacement, for example due to higher morbidity.

Such prosthetic components are offered commercially for hips, knees, shoulders and small joints, based on metallic materials such as titanium or cobalt chromium, wherein these implants perform a two-fold function.

They generally comprise a part that is subject to tribology, which serves as the articular surface, and an osseointegrating part, which grows together with the bone tissue and ensures secure anchoring.

The disadvantages that may occur with the metallic solutions are known.
Metallic abrasion and the resulting negative impact on the human organism
Artifacts in imaging for medical diagnostics
Effects of aging and long-term performance (fatigue, corrosion, and the release of metal ions, which can be toxic)

A rising risk of infection during surgery has also proven to be a general problem with increasing frequency.

In addition to these metallic solutions, moreover there is what is known as an osteochondral implant, which is based on human cartilage and bone tissue and can be implanted into the damaged areas in a minimally invasive procedure. The potential disadvantages of such implants are the high wear, low strength, effects of aging, and inadequate long-term performance.

It was the object of the present invention to provide implants that do not exhibit the disadvantages of the known solutions.

In particular, the implants according to the invention were to have the following properties:
The implant according to the invention was to grow reliably together with the bone tissue of the joint after implantation and ensure sufficiently high anchoring and stability.
The implant according to the invention was to represent a compact unit, from a tribological point of view, together with the remaining joint surface after implantation and be safely and permanently operatively connected to the articulation partner.
During the articulation against a natural cartilage surface, the implant according to the invention was to function reliably, permanently and with low wear.
Ideally, the implant according to the invention was to be designed so as to favor and promote the formation of new, natural cartilage tissue.
During the residence time in the human body, the implant according to the invention was not to cause any adverse health effects. In particular, it was
   not to produce any abrasion particles hazardous to health;
   not to lastingly damage the articulation partner in the interaction therewith, in particular the natural cartilage tissue;
   not to be damaged or destroyed by the biomechanical conditions;
   not to offer conditions favorable for infection-causing bacteria.

The object underlying the invention is achieved by implants having the features of the main claim. Preferred embodiments can be found in the dependent claims.

The invention relates to ceramic-based implants.

In principle, the implant according to the invention comprises at least two different functional units (hereafter also referred to as layers):
The first unit is an osseointegrating ceramic-based unit, which forms a biologically active connection with the bone tissue located beneath the cartilage.
The second unit is likewise a ceramic-based unit, which is connected to the first unit, which are subject to tribology and serve as articular surfaces. The second unit may also serve as a substrate for an optional third unit.
The implant may also comprise further units or layers, and in particular an optional third unit. This optional third unit may be a polymer-based unit, preferably based on PVA or hydrogels, which is fixedly connected to the second unit and ensures the tribological function in the articulation with the natural cartilage. A hydrogel is a polymer that contains water, but is water-insoluble, comprising molecules that are linked chemically, such as by covalent or ionic bonds, or physically, such as by looping polymer chains, to form a three-dimensional network.

The osseointegrative first unit comprises a ceramic material having porous fractions and an open-pored, interconnecting structure. This unit can be produced in the manner known per se, such as by way of
molding processes;
direct freeze foaming processes;
foaming processes;
pore-forming processes based on organic pore inducers.

These structures should be optimally matched to the biological osteogenesis and vascularization processes and ensure optimal osteoconductive properties.

Typical characteristics of such a structure are pore diameters in the order of magnitude between 100 and 1000 μm, and specifically between 300 and 700 μm.

Open porosities are in the order of magnitude between 50% and 90%, and preferably between 60% and 80%. The moduli of elasticity should be in the order of magnitude between 5 and 50 GPa, and ideally in the range of human bone, so as to ensure advantageous mechanical stimulus for forming bone substance.

Additionally, these structures may be coated with osseoinductive coatings of any kind in the manner known per se, whereby the osseointegration effect is further enhanced.

Examples of such coatings that may be used according to the invention include:
bioglasses, specifically the composition 45S5;
hydroxylapatite coatings of any kind, including nanostructured and biomimetically acting layers;
phosphating layers, in particular covalently bound phosphating monolayers;
metallic coatings, in particular based on tantalum or titanium.

The second unit comprises a relatively dense ceramic material, which is characterized in particular by high hardness and strength. This unit is fixedly connected to the first unit. The connection to the first unit can take place, for example, by applying a slip in the green state and subsequent co-sintering, or, depending on the method for producing the first unit, this may be done integrally.

If a third unit is present, the side of the second unit facing the third unit is structured so as to represent an optimal substrate surface for the third unit. This side then must ensure not only a fixed form-locked or force-fit connection, but also dispersion by flowing under the biomechanical load of the third unit, or preferably low shear loading. In particular, it is to be avoided that internal cracking or other damage to the cross-linked polymer occurs.

Specifically, care must be taken during structuring that no sharp edges are created.

If a third unit is present, in a very general sense structures are provided that increase the surface, and thereby ensure a good connection between the second unit and the cartilage or the (optional) third unit, for example by way of adhesion forces or by way of adhesives suitable for medical purposes. For example, defined depressions in the surface, which could appear as a golf ball-like structure, are suitable for this purpose, or undercut or drop-shaped depressions in the range of several μm to mm, which provide optimal support to the polymer materials of the third unit and result in self-stabilization or self-fixation under axial load, or web-like structures.

It is particularly advantageous when, under load, the strength of the connection between the second and optional third units increases. According to the invention, this is achieved by appropriately designed structures on the ceramic second unit.

Ideally, the structure of the second unit, which faces away from the second unit and would face a third unit, has a very low micro-roughness in the range of a few μm, which can be achieved by grinding or polishing, or at least those regions of the structure that are exposed during potential abrasion of the optional third unit.

The structuring of the ceramic material of the second unit can take place by way of injection molding processes (ceramic injection molding, low pressure injection molding) or other molding techniques, or by mechanical processing in the green state or by laser processing, or ultrasound-assisted mechanical processing in the sintered state, or by electrical discharge machining or chemical etching processes.

The ceramic material of the first and second units is preferably an oxide ceramic from the class of the aluminum oxides or zirconium oxides, such as zirconia-reinforced alumina or yttria-stabilized zirconia, but also all variants thereof, or composite materials having the general descriptions ZTA (zirconia-toughened alumina) or ATZ (alumina-toughened zirconia).

According to the invention, non-oxide ceramics, such as materials based on Si₃N₄, may likewise be used.

All listed materials have extremely good tribological properties due to the chemical composition, hardness and strength thereof, are highly tolerant toward damage during implantation, and more than satisfy the biomechanical requirements.

Moreover, without further functionalization they are to a large extent bioinert and tend to prevent the proliferation of bacteria.

All further developments of these material are also advantageous, for example extremely damage-tolerant materials such as rare earth-stabilized dispersoid ceramics made of zirconium oxide comprising fractions of aluminates.

For the optional third unit, three-dimensionally linked polymers have proven to be particularly suitable polymers, which, in addition to the mechanical properties thereof in particular with respect to stiffness and shear loadability, also withstand the biomechanical loads and are similar to natural cartilage material.

Moreover, polymers have the advantage that they can be loaded with functional groups, which allow the physical properties to be set in a targeted manner.

In particular, hydrogels can be used as carriers of biologically active substances that can evoke an antibacterial or chondrogenetic effect. These biologically active substances favor the in vivo formation of endogenous cartilage material.

In this connection, synthetic alginates are also often used in combination with human stem cells, so as to promote the formation of endogenous cartilage tissue. This approach could also be integrated into the third unit.

It is very advantageous when the internal structure of the polymers, in particular of the hydrogels, is configured so that the formation of cartilage is promoted under compressive mechanical stress and with appropriate loading with chondrogenetic substances. These mechanisms also act in the natural cartilage which is dependent on mechanical stimuli.

A possible fixed connection between the second and optional third units can be established by form-locked or force-fit joining processes; however, it is also conceivable, especially in the case of undercut structures, to melt the polymers or generally to apply these in the liquid phase.

The thickness provided according to the invention for the polymer or hydrogel layer is highly dependent on the properties of the polymer. Microcoatings or nanocoatings can be applied or joined to the second unit, or thick layers measuring up to several mm.

Moreover, with respect to the topographical configuration of the articulating surface, it may be extremely advantageous if this surface is adapted to the anatomical and patient-specific conditions, which can be ensured by way of suitable CAD-CAM methods in the manufacturing process.

Furthermore, a perfect fit of the implant from a biomechanical and surgical point of view is extremely advantageous, which can be ensured by a suitable procedure using appropriate and, if necessary, customized instruments.

The implants according to the invention are preferably used for the restoration of joint surfaces in the human body, such as joint surfaces from the shoulder, hip, knee and foot regions. The implants according to the invention are suitable as joint surfaces for local cartilage defects, as partial joint replacements, but also as full endoprostheses. They are particularly suitable in articulations against natural cartilage surfaces, known as hemiprostheses, for example as a shoulder hemiprosthesis, also referred to as a humeral head prosthesis. This is a partial replacement of the shoulder joint, in which the natural shoulder socket (glenoid) is preserved, and only the humeral head is replaced by an endoprosthesis.

In summary, the present invention thus relates to the following:
Ceramic-based implants.
Implants according to item 1, characterized by comprising at least two different functional layers.
Implants according to item 1 or 2, characterized in that the first layer is ceramic-based.
Implants according to one or more of the preceding items, characterized in that the second layer is likewise ceramic-based and is connected to the first layer.
Implants according to one or more of the preceding items, characterized in that the first layer has an osseointegrating property.
Implants according to one or more of the preceding items, characterized in that the second layer ensures the tribological function in the articulation with the natural cartilage.
Implants according to one or more of the preceding items, characterized in that the first layer comprises a ceramic material having porous fractions.
Implants according to one or more of the preceding items, characterized in that the first layer comprises a ceramic material having porous fractions and has an open-pored, interconnecting structure.
Implants according to one or more of the preceding items, characterized in that the first layer has pore diameters in the order of magnitude between 100 and 1000 μm, and preferably between 300 and 700 μm.
Implants according to one or more of the preceding items, characterized in that the first layer has open porosities in the order of magnitude between 50% and 90%, and preferably between 60% and 80%.
Implants according to one or more of the preceding items, characterized in that the moduli of elasticity of the first layer are in the order of magnitude between 5 and 50 GPa, and preferably in the range of human bone.
Implants according to one or more of the preceding items, characterized in that the first layer is coated with osseoinductive coatings.
Implants according to one or more of the preceding items, characterized in that the first layer is coated with osseoinductive coatings, the layer being selected from bioglasses, hydroxylapatite coatings, phosphating layers and/or metallic coatings.
Implants according to one or more of the preceding items, characterized in that the first layer is coated with osseoinductive coatings, the layer being selected from bioglasses, preferably from the bioglass having the composition 45S5, from hydroxylapatite coatings, preferably from nanostructured and biomimetically acting hydroxylapatite layers, from phosphating layers, in particular covalently bound phosphating monolayers, and/or metallic coatings, in particular metallic coatings based on tantalum or titanium.
Implants according to one or more of the preceding items, characterized in that the second layer comprises a relatively dense ceramic material.
Implants according to one or more of the preceding items, characterized in that the second layer has high hardness and strength.
Implants according to one or more of the preceding items, characterized in that the second layer has structures that increase the surface.
Implants according to one or more of the preceding items, characterized in that the second layer has structures that increase the surface, preferably defined depressions in the surface, (golf ball-like structure), undercut or drop-shaped depressions in the range of several μm to mm, or web-like structures.
Implants according to one or more of the preceding items, characterized in that the second layer has a side facing away from the second layer which has a very low micro-roughness in the range of a few μm.
Implants according to one or more of the preceding items, characterized in that the ceramic material of the first and second layers comprises an oxide ceramic from the class of aluminum oxides or zirconium oxides or a non-oxide ceramic, such as materials based on Si₃N₄.
Use of the implants according to one or more of the preceding items for the restoration of joint surfaces in the human body, such as joint surfaces from the shoulder, hip, knee and foot regions.
Use of the implants according to one or more of the preceding items as joint surfaces for local cartilage defects, as partial joint replacements, but also as full endoprostheses.
Use of the implants according to one or more of the preceding items in articulations against natural cartilage surfaces.

The invention is in particular characterized by the following embodiments:

### EMBODIMENT 1

A ceramic-based implant.

### EMBODIMENT 2

The implant according to embodiment 1, characterized by comprising at least two different functional layers.

### EMBODIMENT 3

The implant according to embodiment 1 or 2, characterized in that the first layer is ceramic-based.

### EMBODIMENT 4

The implant according to one or more of the preceding embodiments, characterized in that the second layer is likewise ceramic-based and is connected to the first layer.

### EMBODIMENT 5

The implant according to one or more of the preceding embodiments, characterized in that the first layer has an osseointegrating property.

### EMBODIMENT 6

The implant according to one or more of the preceding embodiments, characterized in that the second layer ensures the tribological function in the articulation with the natural cartilage.

### EMBODIMENT 7

The implant according to one or more of the preceding embodiments, characterized in that the first layer comprises a ceramic material having porous fractions.

### EMBODIMENT 8

The implant according to one or more of the preceding embodiments, characterized in that the first layer comprises a ceramic material having porous fractions and has an open-pored, interconnecting structure.

### EMBODIMENT 9

The implant according to one or more of the preceding embodiments, characterized in that the first layer has pore diameters in the order of magnitude between 100 and 1000 μm, and preferably between 300 and 700 μm.

### EMBODIMENT 10

The implant according to one or more of the preceding embodiments, characterized in that the first layer has open porosities in the order of magnitude between 50% and 90%, and preferably between 60% and 80%.

### EMBODIMENT 11

The implant according to one or more of the preceding embodiments, characterized in that the moduli of elasticity of the first layer are in the order of magnitude between 5 and 50 GPa, and preferably in the range of human bone.

### EMBODIMENT 12

The implant according to one or more of the preceding embodiments, characterized in that the first layer is coated with osseoinductive coatings.

### EMBODIMENT 13

The implant according to one or more of the preceding embodiments, characterized in that the first layer is coated with osseoinductive coatings, the layer being selected from bioglasses, hydroxylapatite coatings, phosphating layers and/or metallic coatings.

### EMBODIMENT 14

The implant according to one or more of the preceding embodiments, characterized in that the first layer is coated with osseoinductive coatings, the layer being selected from bioglasses, preferably from the bioglass having the composition 45S5, from hydroxylapatite coatings, preferably from nanostructured and biomimetically acting hydroxylapatite layers, from phosphating layers, in particular covalently bound phosphating monolayers, and/or metallic coatings, in particular metallic coatings based on tantalum or titanium.

### EMBODIMENT 15

The implant according to one or more of the preceding embodiments, characterized in that the second layer comprises a relatively dense ceramic material.

### EMBODIMENT 16

The implant according to one or more of the preceding embodiments, characterized in that the second layer has high hardness and strength.

### EMBODIMENT 17

The implant according to one or more of the preceding embodiments, characterized in that the second layer has structures that increase the surface.

### EMBODIMENT 18

The implant according to one or more of the preceding embodiments, characterized in that the second layer has structures that increase the surface, preferably defined depressions in the surface, (golf ball-like structure), undercut or drop-shaped depressions in the range of several μm to mm, or web-like structures.

### EMBODIMENT 19

The implant according to one or more of the preceding embodiments, characterized in that the second layer has a side facing away from the second layer which has a very low micro-roughness in the range of a few μm.

### EMBODIMENT 20

The implant according to one or more of the preceding embodiments, characterized in that the ceramic material of the first and second layers comprises an oxide ceramic from the class of aluminum oxides or zirconium oxides or a non-oxide ceramic, such as materials based on Si₃N₄.

In a preferred embodiment 21, the present invention relates to the use of the implant according to one or more of the preceding embodiments for the restoration of joint surfaces in the human body, such as joint surfaces from the shoulder, hip, knee and foot regions.

### EMBODIMENT 22

Use of the implant according to one or more of the preceding embodiments as joint surfaces for local cartilage defects, as partial joint replacements, but also as full endoprostheses.

### EMBODIMENT 23

Use of the implant according to one or more of the preceding embodiments in articulations against natural cartilage surfaces.

1. A ceramic-based implant, characterized by comprising at least two different functional layers, the first layer being ceramic-based, and the second layer likewise being ceramic-based and connected to the first layer.

2. The implant according to claim 1, wherein the first layer has pore diameters in the order of magnitude between 100 and 1000 μm, and preferably between 300 and 700 μm.

3. The implant according to claim 1, wherein the first layer has open porosities in the order of magnitude between 50% and 90%, and preferably between 60% and 80%.

4. The implant according to claim 1, wherein the first layer is coated with osseoinductive coatings, the layer being selected from bioglasses, hydroxylapatite coatings, phosphating layers and/or metallic coatings.

5. The implant according to claim 1, wherein the first layer is coated with osseoinductive coatings, the layer being selected from bioglasses, preferably from the bioglass having the composition 45S5, from hydroxylapatite coatings, preferably from nanostructured and biomimetically acting hydroxylapatite layers, from phosphating layers, in particular covalently bound phosphating monolayers, and/or metallic coatings, in particular metallic coatings based on tantalum or titanium.

6. The implant according to claim 1, wherein the second layer has structures that increase the surface, preferably defined depressions in the surface, (golf ball-like structures), undercut or drop-shaped depressions in the range of several μm to mm, or web-like structures.

7. The implant according to claim 1, wherein the second layer has a side facing away from the second layer which has a very low micro-roughness in the range of a few μm.

8. The implant according to claim 1, wherein the ceramic material of the first and second layers comprises an oxide ceramic from the class of aluminum oxides or zirconium oxides or a non-oxide ceramic, such as materials based on Si3N4.

9. Use of the implant according to claim 1 for the restoration of joint surfaces in the human body, such as joint surfaces from the shoulder, hip, knee and foot regions.

10. Use of the implant according to claim 1 as joint surfaces for local cartilage defects, as partial joint replacements, but also as full endoprostheses.
